(19) United States
(12) Patent Application Publication
(10) Pub. No.: US 2011/0021630 A1
(43) Pub. Date: Jan. 27, 2011

(54) METHOD FOR TREATING WOUNDS BY ADMINISTERING FULLERENES

(76) Inventors: Christopher L. Kepley, Ringgold, VA (US); Robert P. Lenk, Danville, VA (US); Stephen R. Wilson, Danville, VA (US); Zhiguo Zhou, Winston-Salem, NC (US)

Correspondence Address: BUCHANAN, INGERSOLL & ROONEY PC, POST OFFICE BOX 1404, ALEXANDRIA, VA, 22313-1404, US

(21) Appl. No.: 12/921,143

(22) PCT Filed: Mar. 3, 2009
(86) PCT No.: PCT/US09/01329

§ 371 Date: Sep. 3, 2010

Related U.S. Application Data
(60) Provisional application No. 61/033,320, filed on Mar. 3, 2008.

(51) Int. Cl. A61K 31/225; C07C 69/34; A61P 17/02

(52) U.S. Cl. 514/547; 560/190

(57) ABSTRACT

Disclosed herein are methods for treating wounds. In one embodiment, the method comprises: administering to a subject in need thereof a therapeutically effective amount of a synthetically modified fullerene of the formula Zₘ—F—Yₙ wherein F is a fullerene of formula Cₚ or X@Cₚ, the fullerene having two opposing poles and an equatorial region; Cₚ represents a fullerene cage having p carbon atoms, and X@C_{P} represents such a fullerene cage having a chemical group X within the cage; Z and Y are positioned near respective opposite poles of Cₚ; m=1-5 and Z is a hydrophilic, lipophilic, or amphiphilic chemical moiety; n=1-5 and Y is a lipophilic chemical moiety; p=60-200 and p is an even number; and X, if present, represents one or more metal atoms within the fullerene (F), optionally in the form of a trinitride of formula Gᵢ₌₁₋₃Hₖ₌₃₋ᵢN in which G and H are metal atoms.

### BACKGROUND

Wounds are internal or external bodily injuries or lesions caused by physical means, such as mechanical, chemical viral, bacterial, or thermal means, which disrupt the normal continuity of structures. Such bodily injuries include contusions, wounds in which the skin is unbroken, incisions, wounds in which the skin is broken by a cutting instrument, and lacerations, wounds in which the skin is broken by a dull or blunt instrument. Wounds may be caused by accidents or by surgical procedures.

Wound healing consists of a series of processes whereby injured tissue is repaired, specialized tissue is regenerated, and new tissue is reorganized. Wound healing consists of three major phases: a) an inflammation phase (about 0-3 days), b) a cellular proliferation phase (about 3-12 days), and (c) a remodeling phase (about 3 days-6 months).

During the inflammation phase, platelet aggregation and clotting form a matrix which traps plasma proteins and blood cells to induce the influx of various types of cells. During the cellular proliferation phase, new connective or granulation tissue and blood vessels are formed. During the remodeling phase, granulation tissue is replaced by a network of collagen and elastin fibers leading to the formation of scar tissue.

When cells are injured or killed as a result of a wound, a wound healing step is desirable to resuscitate the injured cells and produce new cells to replace the dead cells. The healing process requires the reversal of cytotoxicity, the suppression of inflammation, and the stimulation of cellular viability and proliferation. Wounds require low levels of oxygen in the initial stages of healing to suppress oxidative damage and higher levels of oxygen in the later stages of healing to promote collagen formation by fibroblasts.

Mammalian cells are continuously exposed to activated oxygen species such as superoxide (O₂—), hydrogen peroxide (H₂O₂), hydroxyl radical (OH.), and singlet oxygen (¹O₂). In vivo, these reactive oxygen intermediates are generated by cells in response to aerobic metabolism, catabolism of drugs and other xenobiotics, ultraviolet and x-ray radiation, and the respiratory burst of phagocytic cells (such as white blood cells) to kill invading bacteria such as those introduced through wounds. Hydrogen peroxide, for example, is produced during respiration of most living organisms especially by stressed and injured cells.

These active oxygen species can injure cells. An important example of such damage is lipid peroxidation which involves the oxidative degradation of unsaturated lipids. Lipid peroxidation is highly detrimental to membrane structure and function and can cause numerous cytopathological effects. Cells defend against lipid peroxidation by producing radical scavengers such as superoxide dismutase, catalase, and peroxidase. Injured cells have a decreased ability to produce radical scavengers. Excess hydrogen peroxide can react with DNA to cause backbone breakage, produce mutations, and alter and liberate bases. Hydrogen peroxide can also react with pyrimidines to open the 5,6-double bond, which reaction inhibits the ability of pyrimidines to hydrogen bond to complementary bases, see for example, Kondo et al., Sonolysis, Radiolysis, and Hydrogen Peroxide Photolysis of Pyrimidine Derivatives in Aqueous Solutions: A Spin-Trapping Study, Radiation Research, 116(1):56-73 (October 1988). Such oxidative biochemical injury can result in the loss of cellular membrane integrity, reduced enzyme activity, changes in transport kinetics, changes in membrane lipid content, and leakage of potassium ions, amino acids, and other cellular material.

There is a need for effective wound healing pharmaceutical compositions. Disclosed herein are methods for treating wounds in a subject in need thereof by administering to the subject a therapeutically effective amount of a synthetically modified fullerene, which is described hereinbelow. These fullerenes can be administered to the subject in a pharmaceutical composition.

Fullerene molecules are a family of carbon allotropes that comprise closed cages of generally 60 to 200 carbon atoms and may also include chemical moieties attached to the exterior or incorporated within the cage. Fullerenes can be in the form of a hollow sphere, ellipsoid, or tube. The most common fullerene to date is the C₆₀ Buckminsterfullerene (IUPAC name (C60-Ih)[5,6]fullerene). Another fairly common buckminsterfullerene is C₇₀, but fullerenes with 72, 76, 84 and even up to 100 carbon atoms are commonly obtained. Fullerene molecules can contain as few as 20 or more than 500 carbon atoms. Fullerenes may enclose one or more atoms such as metal atoms, or other small chemical groups, inside the carbon cage; such fullerenes are sometimes called endohedral fullerenes. Fullerenes may also be modified or derivatized to include chemical functional groups attached to the surface of the carbon cage.

### SUMMARY

Disclosed herein are methods for treating wounds. In one embodiment, the method comprises: administering to a subject in need thereof a therapeutically effective amount of a synthetically modified fullerene of the formula Zₘ—F—Yₙ, wherein F is a fullerene of formula Cₚ or X@Cₚ, the fullerene having two opposing poles and an equatorial region; Cₚ represents a fullerene cage having p carbon atoms, and X@Cₚ represents such a fullerene cage having a chemical group X within the cage; Z and Y are positioned near respective opposite poles of Cₚ; m=1-5 and Z is a hydrophilic, lipophilic, or amphiphilic chemical moiety; n=1-5 and Y is a lipophilic chemical moiety; p=60-200 and p is an even number; and X, if present, represents one or more metal atoms within the fullerene (F), optionally in the form of a trinitride of formula Gᵢ₌₁₋₃Hₖ₌₃₋ᵢN in which G and H are metal atoms.

Also, disclosed herein is a kit for treating wounds comprising: administering to a subject in need thereof a therapeutically effective amount of a synthetically modified fullerene of the formula Zₘ—F—Yₙ wherein F is a fullerene of formula Cₚ or X@Cₚ, the fullerene having two opposing poles and an equatorial region; Cₚ represents a fullerene cage having p carbon atoms, and X@Cₚ represents such a fullerene cage having a chemical group X within the cage; Z and Y are positioned near respective opposite poles of Cₚ; m=1-5 and Z is a hydrophilic, lipophilic, or amphiphilic chemical moiety; n=1-5 and Y is a lipophilic chemical moiety; p=60-200 and p is an even number; and X, if present, represents one or more metal atoms within the fullerene (F), optionally in the form of a trinitride of formula Gᵢ₌₁₋₃Hₖ₌₃₋ᵢN in which G and H are metal atoms.

### BRIEF DESCRIPTION OF THE DRAWINGS

FIG. 1 illustrates non-limiting examples of synthetically modified fullerenes with any combination of hydrophilic, lipophilic, or amphiphilic chemical moieties.

FIG. 2 illustrates additional non-limiting examples of synthetically modified fullerenes with any combination of hydrophilic, lipophilic, or amphiphilic chemical moieties.

FIG. 3 illustrates additional non-limiting examples of derivatized fullerenes with any combination of hydrophilic, lipophilic, or amphiphilic chemical moieties.

FIG. 4 illustrates additional non-limiting examples of derivatized fullerenes with any combination of hydrophilic, lipophilic, or amphiphilic chemical moieties.

FIG. 5 illustrates the effectiveness of compound 5 (ALM) in inhibiting the initial inflammatory response in the wound healing process in mice.

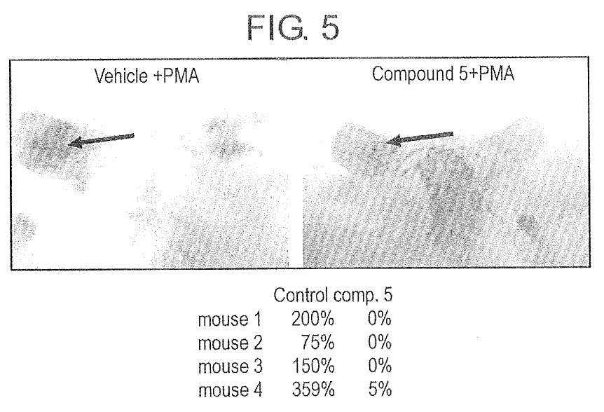

FIG. 6 illustrates the effectiveness of compound 5 (ALM) in accelerating the wound healing process in mice.

### DETAILED DESCRIPTION

In accordance with this detailed description, the following abbreviations and definitions apply. It must be noted that as used herein, the singular forms “a”, “an”, and “the” include plural referents unless the context clearly dictates otherwise. Thus, for example, reference to “compounds” includes a plurality of such compounds and reference to “the dosage” includes reference to one or more dosages and equivalents thereof known to those skilled in the art, and so forth.

The publications discussed in this disclosure are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates, which may need to be independently confirmed.

Unless otherwise stated, the following terms used in the specification and claims have the meanings given below:

“Wound” or “wounds” as used herein refers to any injury or opening in tissue. Wounds are internal or external bodily injuries or lesions caused by physical means, such as mechanical, chemical viral, bacterial, or thermal means, which disrupt the normal continuity of structures. Such bodily injuries include contusions, wounds in which the skin is unbroken, incisions, wounds in which the skin is broken by a cutting instrument, and lacerations, wounds in which the skin is broken by a dull or blunt instrument. Wounds may be caused by accidents or by surgical procedures.

Wound healing consists of a series of processes whereby injured tissue is repaired, specialized tissue is regenerated, and new tissue is reorganized. Wound healing consists of three major phases: a) an inflammation phase (about 0-3 days), b) a cellular proliferation phase (about 3-12 days), and (c) a remodeling phase (about 3 days-6 months).

During the inflammation phase, platelet aggregation and clotting form a matrix which traps plasma proteins and blood cells to induce the influx of various types of cells. During the cellular proliferation phase, new connective or granulation tissue and blood vessels are formed. During the remodeling phase, granulation tissue is replaced by a network of collagen and elastin fibers leading to the formation of scar tissue.

When cells are injured or killed as a result of a wound, a wound healing step is desirable to resuscitate the injured cells and produce new cells to replace the dead cells. The healing process requires the reversal of cytotoxicity, the suppression of inflammation, and the stimulation of cellular viability and proliferation. Wounds require low levels of oxygen in the initial stages of healing to suppress oxidative damage and higher levels of oxygen in the later stages of healing to promote collagen formation by fibroblasts.

Mammalian cells are continuously exposed to activated oxygen species such as superoxide (O₂—), hydrogen peroxide (H₂O₂), hydroxyl radical (OH.), and singlet oxygen (¹O₂). In vivo, these reactive oxygen intermediates are generated by cells in response to aerobic metabolism, catabolism of drugs and other xenobiotics, ultraviolet and x-ray radiation, and the respiratory burst of phagocytic cells (such as white blood cells) to kill invading bacteria such as those introduced through wounds. Hydrogen peroxide, for example, is produced during respiration of most living organisms especially by stressed and injured cells.

These active oxygen species can injure cells. An important example of such damage is lipid peroxidation which involves the oxidative degradation of unsaturated lipids. Lipid peroxidation is highly detrimental to membrane structure and function and can cause numerous cytopathological effects. Cells defend against lipid peroxidation by producing radical scavengers such as superoxide dismutase, catalase, and peroxidase. Injured cells have a decreased ability to produce radical scavengers. Excess hydrogen peroxide can react with DNA to cause backbone breakage, produce mutations, and alter and liberate bases. Hydrogen peroxide can also react with pyrimidines to open the 5,6-double bond, which reaction inhibits the ability of pyrimidines to hydrogen bond to complementary bases, see for example, Kondo et al., Sonolysis, Radiolysis, and Hydrogen Peroxide Photolysis of

Pyrimidine Derivatives in Aqueous Solutions: A Spin-Trapping Study, Radiation Research, 116(1):56-73 (October 1988). Such oxidative biochemical injury can result in the loss of cellular membrane integrity, reduced enzyme activity, changes in transport kinetics, changes in membrane lipid content, and leakage of potassium ions, amino acids, and other cellular material.

Intimation is a nonspecific response caused by several kinds of injury, including penetration of the host by infectious agents. The distinguishing feature of inflammation is dilation and increased permeability of minute blood vessels. Direct injury, such as that caused by toxins elaborated by microorganisms, leads to destruction of vascular endothelium and increased permeability to plasma proteins, especially in the venules and venular capillaries. Mediators of secondary injury are liberated from the site of direct injury. As a result, gaps form between vascular endothelial cells through which plasma proteins escape. Granulocytes, monocytes, and erythrocytes may also leave vascular channels. Mediators of secondary injury include unknown substances and histamine, peptides (kinins), kinin-forming enzymes (kininogenases), and a globulin permeability factor. These mediators are blocked from action by antihistamines and sympathoamines, and are most pronounced in effect on venules, although lymphvascular endothelium also becomes more porous as a part of secondary injury.

The beneficial effect of the inflammatory response is the production of: (1) leukocytes in great numbers; (2) plasma proteins, nonspecific and specific humoral agents, fibrinogen that on conversion to fibrin aids in localization of the infectious process while acting as a matrix for phagocytosis; and (3) increased blood and lymph flow that dilutes and flushes toxic materials while causing a local increase in temperature.

In the early stages of inflammation, the exudate is alkaline and neutrophilic polymorphonuclear leukocytes predominate. As lactic acid accumulates, presumably from glycolysis, the pH drops and macrophages become the predominant cell type. Lactic acid and antibodies in the inflammatory exudate may inhibit parasites, but the major anti-infectious effect of the inflammatory response is attributable to phagocytic cells.

The inflammatory response consists of three successive phases: (a) increased vascular permeability with resulting edema and swelling, (b) cellular infiltration and phagocytoses, and (c) proliferation of the fibroblasts, synthesizing new connective tissue to repair the injury. A large number of so-called mediators of inflammation have been implicated in the inflammatory process primarily in terms of their capacity to induce vasodilation and increase permeability.

The initial increase in capillary permeability and vasodilation in an inflamed joint is followed by an increase in metabolism of the joint tissues. Leakage of fibrinogen into the wound, where proteolytic enzymes convert it into fibrin, establishes a capillary and lymphatic blockade. The concentrations of components of the ground substance of connective tissue collagen, mucopolysaccharides, glycoproteins, and nonfibrous proteins are greatly increased during this process. As the exudative phase of the inflammation subsides, the fibroblast is found to be the dominant cell in the wounded zone. It first proliferates, then synthesizes extracellular material, including new collagen fibers and acid mucopolysaccharides, which are laid down to form the new tissue matrix.

The inflammatory phenomenon includes fenestration of the microvasculature, leakage of the elements of blood into the interstitial spaces, and migration of leukocytes into the inflamed tissue. On a macroscopic level, this is usually accompanied by the familiar clinical signs of erythema, edema tenderness (hyperalgesia), and pain. During this complex response, chemical mediators such as histamine, 5-hydroxytryptamine (5-HT), slow-reacting substance of anaphylaxis (SRS-A), various chemotactic factors, bradykinin, and prostaglandins are liberated locally. Phagocytic cells migrate into the area, and cellular lysosomal membranes may be ruptured, releasing lyric enzymes. All these events may contribute to the inflammatory response.

According to various embodiments described herein, disclosed are methods for treating wounds in a subject in need thereof by administering a therapeutically effective amount of fullerenes. More particularly, these methods disclose that the fullerenes inhibit the inflammation response during the wound healing process and accelerate the wound healing process such that the wound is effectively healed. Even more particularly, the fullerenes described herein reduce injury to mammalian cells and increase the resuscitation rate of injured mammalian cells.

Tissue damage resulting from chemical, mechanical, and biological injury, or from interrupted blood flow and reperfusion, is often life threatening. The subsequent tissue response involves an intricate series of events including inflammation, oxidative stress, immune cell recruitment, and cell survival, proliferation, migration, and differentiation. While normally beneficial in the physiological defense process, inflammation and oxidative stress can become harmful if they do not resolve in time. Organ damage can occur in response to an inadequate supply of oxygen (hypoxia), usually caused by blood vessel constriction or obstruction (ischemia). Under normal physiological conditions, oxygenation levels and sensitivity to hypoxia differ among the various organs. Since short periods of ischemia and reperfusion (ischemia/reperfusion, or UR) cause extensive damage, the goal of the survival response is to maintain tissue viability. As a result, the hypoxia response requires optimal revascularization for efficient recovery.

The generation of free radicals during the process of wound healing can cause acute secondary damage such as ischemia/reperfusion injury and cell death. The fullerenes described herein can inhibit free radicals. Moreover, these fullerenes can not only inhibit the initial inflammatory response during wound healing but dramatically accelerate the wound healing process.

In one embodiment a pharmaceutical composition comprising water soluble fullerenes in a form suitable for intravenous delivery into the body of a subject where inhibition of the inflammatory response of the wound healing process is desired. In another embodiment a pharmaceutical composition comprising water soluble fullerenes in a form suitable for topical application directly to an anatomical area of the body of a subject where inhibition of the inflammatory response of the wound healing process is desired. In one embodiment a pharmaceutical composition comprising water soluble fullerenes in a form suitable for a cream form that can be applied directly to an anatomical area of the body of a subject where inhibition of the inflammatory response of the wound healing process is desired. In another embodiment a pharmaceutical composition comprising water soluble fullerenes can be added to other dermatological products (e.g., gels, soaps, etc.) that can be applied directly to an anatomical area of the body of a subject where inhibition of the inflammatory response of the wound healing process is desired.

In one embodiment a pharmaceutical composition comprising water soluble fullerenes in a form suitable for intravenous delivery into the body of a subject where acceleration of the wound healing process is desired. In another embodiment a pharmaceutical composition comprising water soluble fullerenes in a form suitable for topical application directly to an anatomical area of the body of a subject where acceleration of the wound healing process is desired. In one embodiment a pharmaceutical composition comprising water soluble fullerenes in a form suitable for a cream form that can be applied directly to an anatomical area of the body of a subject where acceleration of the wound healing process is desired. In another embodiment a pharmaceutical composition comprising water soluble fullerenes can be added to other dermatological products (e.g., gels, soaps, etc.) that can be applied directly to an anatomical area of the body of a subject where acceleration of the wound healing process is desired.

“Fullerene” or “fullerene molecule” as used herein refers to certain synthetically modified fullerene molecules as described herein, including amphiphilic or lipophilic synthetically modified fullerenes of the formula Zₘ—F—Yₙ; and hydrophilic or amphiphilic synthetically modified fullerenes of the formula Z′m-F—Y′n. The fullerenes comprise closed cages of 60 to 200 carbon atoms which may also include chemical moieties attached to the exterior and/or incorporated within the cage.

The amphiphilic or lipophilic synthetically modified fullerene molecules are described in copending U.S. patent application Ser. No. 2/073,230, U.S. Patent Application Publication No. 2008-0213324-A1 filed Mar. 3, 2008, entitled “AMPHIPHILIC OR LIPOPHILIC POLAR FUNCTIONALIZED FULLERENES AND THEIR USES,” the entire disclosure of which is incorporated by reference herein.

The amphiphilic or lipophilic synthetically modified fullerene molecules as described in the copending application include fullerenes that have an aspect ratio 1, with an equatorial band and two opposing poles, and comprise an adduct at one or both poles.

In one embodiment, the amphiphilic or lipophilic synthetically modified fullerene has the formula

Zₘ—F—Yₙ;
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage.
Z and Y are positioned near respective opposite poles of Cₚ;
m=1-5 and Z is a hydrophilic, lipophilic, or amphiphilic chemical moiety;
n=1-5 and Y is a lipophilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms.

In exemplary variations p is an even number between 60 and 120, with p=60-96 being more common and p=60 or p=70 being preferred. The synthetically modified fullerene can be arranged wherein each chemical moiety Z is composed of formula AᵣB in which A is a hydrophilic, lipophilic or amphiphilic chemical moiety, r=1-4, and B is a chemical linker connecting said A to the fullerene, and each chemical moiety Y is composed of formula DE, in which E is a lipophilic chemical moiety, v=1-4, and D is a chemical linker connecting the lipophilic chemical moiety to the fullerene.

The amphiphilic or lipophilic synthetically modified fullerene can be a prolate ellipsoid shaped fullerene having a major axis such that said poles are located at opposing ends of the major axis of the prolate ellipsoid fullerene. Alternatively, the fullerene can be spheroid with opposing poles defined by an axis through opposing carbon rings. Z and Y can configured such that when the molecule is contacted with a lipid bilayer in an aqueous medium, the equatorial region of F is selectively located within or in close proximity to the phospholipid bilayer. The molecule can be configured so that in an extended configuration has an aspect ratio of about 2.1 to 15, and a diameter less than about 2 nm. Such configurations are preferred configurations for incorporation of the molecules into lipid bilayers.

In another embodiment, the amphiphilic or lipophilic synthetically modified fullerene molecule has the formula Z(Cₚ)Y wherein: p=60-200 carbons, preferably p=60 or 70; Y is a lipophilic moiety covalently connected to Cₚ, optionally through a linking group, at or near a pole thereof, and wherein Z is a lipophilic moiety, amphiphilic moiety, or a hydrophilic moiety covalently connected to Cₚ, optionally through a linking group, at or near a pole opposite to said Y; and, wherein said lipophilic moiety Y is capable of anchoring the synthetic fullerene molecule to a lipid membrane;

In another embodiment, the amphiphilic or lipophilic synthetically modified fullerene molecule has the formula Z(Cₚ)Y wherein: p=60-200 carbons, preferably p=60 or 70; Y is a lipophilic moiety covalently connected to Cₚ, optionally through a linking group, at or near a pole thereof, and wherein Z is a hydrophilic moiety covalently connected to Cₚ, optionally through a linking group, at or near a pole opposite to said Y; and, wherein said lipophilic moiety Y is capable of anchoring the synthetic fullerene molecule to a lipid membrane.

In another embodiment, the amphiphilic or lipophilic synthetically modified fullerene molecule has the formula Z(C₇₀)Y; wherein Y is a lipophilic moiety covalently connected to C₇₀, optionally through a linking group, at or near a pole thereof, and wherein Z is a lipophilic moiety, amphiphilic moiety, or a hydrophilic moiety covalently connected to C₇₀, optionally through a linking group, at or near a pole opposite to said Y; and, wherein said lipophilic moiety Y is capable of anchoring the synthetic fullerene molecule to a lipid membrane.

In another embodiment, the amphiphilic or lipophilic synthetically modified fullerene molecule has the formula Z(C₇₀)Y wherein: Y is a lipophilic moiety covalently connected to Cₚ, optionally through a linking group, at or near a pole thereof, and wherein Z is a hydrophilic moiety covalently connected to Cₚ, optionally through a linking group, at or near a pole opposite to said Y; and, wherein said lipophilic moiety Y is capable of anchoring the synthetic fullerene molecule to a lipid membrane.

In another embodiment the amphiphilic or lipophilic synthetically modified fullerene molecule can have the formula Zₘ—F—Yₙ wherein:
F is a fullerene of formula Cₚ having p=60-200 carbons, preferably p=60 or 70;
m=1-5 such that each Z is a group AᵣBₛ in which r=1-4, s=1-4, and A is one or more hydrophilic or charged group bonded to the fullerene through one or more linker B;
n=1-5 and each Y is a group DₜE, in which t=1-4, v=1-4 and E is one or more lipophilic group bonded to the fullerene through one or more linker D; and,
X and Y are positioned at or near opposite poles of F.

In certain embodiments the amphiphilic or lipophilic synthetically modified fullerene has a geometrical configuration capable of causing the fullerene molecule to locate within phospholipid bilayers of a cell such that a radical scavenging zone near the equatorial band of the fullerene is situated within or in close proximity to the phospholipid bilayer.

A plurality of such synthetically modified fullerene molecules can be uniformly dispersed in phospholipids, such as in liposomes. The amphipathic fullerene molecules described herein do not generally form vesicles by themselves, but require membrane-forming phospholipids in mole ratios greater than 1:1 (lipid:fullerene adduct) to form vesicles.

The methods described herein also encompass hydrophilic or amphiphilic synthetically modified fullerenes of the formula

Z′ₘ—F—Y′ₙ;
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage;
Z′ and Y′ are positioned near respective opposite poles of Cₚ;
m=1-5 and Z′ is a hydrophilic, lipophilic, or amphiphilic chemical moiety;
n=1-5 and Y′ is a hydrophilic or amphiphilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms.

In exemplary variations p is an even number between 60 and 120, with p=60-96 being more common and p=60 or p=70 being preferred. The fullerene can be arranged wherein each chemical moiety Z′ is composed of formula A′ᵣB in which A′ is a hydrophilic, lipophilic or amphiphilic chemical moiety, r=1-4, and B is a chemical linker connecting said A′ to the fullerene, and each chemical moiety Y′ is composed of formula DE′ᵥ in which E′ is a hydrophilic or amphiphilic chemical moiety and, v=1-4, and D is a chemical linker connecting the chemical moiety Y′ to the fullerene.

In another embodiment, the hydrophilic or amphiphilic synthetically modified fullerene molecule has the formula Z′(Cₚ)Y′ wherein: p=60-200 carbons, preferably p=60 or 70; Y′ is a hydrophilic or amphiphilic moiety covalently connected to Cₚ, optionally through a linking group, at or near a pole thereof, and wherein Z′ is a hydrophilic or amphiphilic moiety covalently connected to Cₚ, optionally through a linking group, at or near a pole opposite to said Y′.

In exemplary embodiments, Z′ and Y′ are both amphiphilic; Z′ and Y′ are both hydrophilic; or one of Z′ and Y′ is amphiphilic while the other is hydrophilic. In other embodiments, Z′ is lipophilic and Y′ is hydrophilic or amphiphilic.

In another embodiment, the hydrophilic or amphiphilic synthetically modified fullerene molecule has the formula E(C₇₀)Y; wherein Y′ is a hydrophilic or amphiphilic moiety covalently connected to C₇₀, optionally through a linking group, at or near a pole thereof; and wherein Z′ is a hydrophilic or amphiphilic moiety covalently connected to C₇₀, optionally through a linking group, at or near a pole opposite to said Y′.

Suitable fullerenes are also described in the following co-pending PCT applications filed concurrently herewith: Attorney Docket No. 1034136-000062, entitled “USING FULLERENES TO ENHANCE AND STIMULATE HAIR GROWTH;” Docket No. 1034136-000063, entitled “METHOD FOR TREATING PRURITUS BY ADMINISTERING FULLERENES;” Attorney Docket No. 1034136-000064, entitled “FULLERENE THERAPIES FOR INFLAMMATION;” and Attorney Docket No. 1034136-000065, entitled “METHOD FOR INHIBITING THE BUILD-UP OF ARTERIAL PLAQUE;” the entire disclosures of which are incorporated by reference herein.

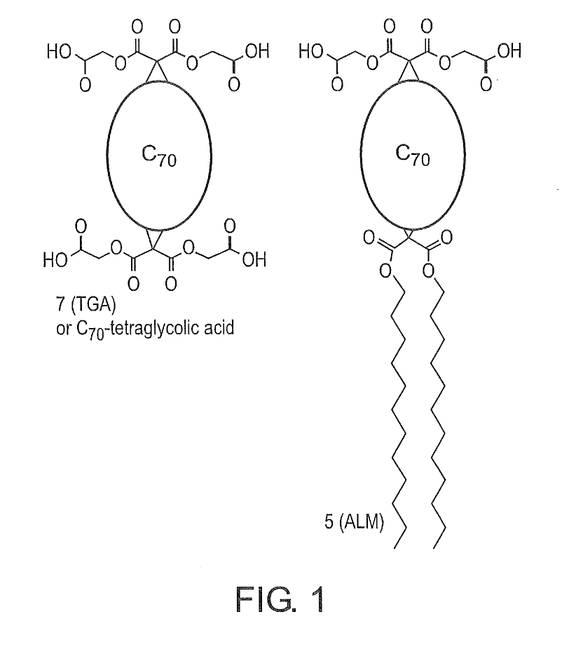

In certain embodiments, the fullerene comprises any one or more of the compounds set forth in the figures. In an exemplary embodiment, the fullerene is compound 5 and/or compound 7 (illustrated in FIG. 1). In the present examples, compound 5 comprises C₇₀.

The terms “treating,” “treatment,” and the like are used herein to generally mean obtaining a desired pharmacological and physiological effect, and refer to complete elimination as well as to any clinically or quantitatively measurable reduction in the condition for which the subject is being treated. In various embodiments, described herein an effective way of “treating” or an effective “treatment” for wound healing is one in which upon the administration of a therapeutically effective pharmaceutical composition comprising fullerenes described herein, the inhibition of the inflammatory response of the wound healing process and the acceleration of the wound healing process can be achieved such that the wound is effectively healed. “Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. More specifically, the fullerenes described herein which are used to treat a subject with a wound are provided in a therapeutically effective amount to inhibit the inflammatory response of the wound healing process and to accelerate the wound healing process such that the wound is effectively healed. Generally, “treatment” as used herein also includes the fullerenes described herein that prevent the disorder (i.e., inhibit the onset or occurrence of the disorder and/or cause the clinical symptoms of the disorder not to develop in a mammal that may be exposed to or predisposed to the disorder but does not yet experience or display symptoms of the disorder); inhibit the disorder (i.e., arrest or reduce the development of the disorder or its clinical symptoms); or relieve the disorder (i.e., cause regression of the disorder or its clinical symptoms). Subjects in need of treatment include those already with a wound.

A “subject in need thereof” refers to any subject or individual who could benefit from the method of treatment described herein. In certain embodiments, a subject in need thereof is a subject having a wound (i.e., on a localized or widespread portion of the subject's body). The “subject in need thereof” refers to a vertebrate, preferably a mammal. Mammals include, but are not limited to, humans, other primates, rodents (i.e., mice, rats, and hamsters), farm animals, sport animals and pets. In one embodiment, the subject is a mammal such as a human. In certain embodiments, the methods find use in experimental animals, in veterinary application, and/or in the development of animal models for disease.

As used herein, the term “administering” or “introducing” fullerenes to a subject means providing the fullerenes to a subject. Methods of administering fullerenes to subjects are well known to those of ordinary skill in the art and include, but are not limited to, oral, intravenous, intramuscular, parenteral, or local administration. Modes of administration can also include delivery via a controlled release and/or controlled release drug delivery formulation and/or device.

“Sustained release” refers to release of a drug or an active metabolite thereof into the systemic circulation over a prolonged period of time relative to that achieved by oral administration of a conventional formulation of the drug.

“Controlled release” is a zero order release; that is, the drug releases over time irrespective of concentration. Single, multiple, continuous or intermittent administration can be effected.

“Orally delivered drugs” refer to drugs which are administered to an animal in an oral form, preferably, in a pharmaceutically acceptable diluent. Oral delivery includes ingestion of the drug as well as oral gavage of the drug.

“Therapeutic or prophylactic blood concentrations” refers to systemic exposure to a sufficient concentration of a drug or an active metabolite thereof over a sufficient period of time to effect disease therapy or to prevent the onset or reduce the severity of a disease in the treated animal.

“Optional” or “optionally” means that the subsequently described event or circumstance may, but need not, occur, and that the description includes instances where the event or circumstance occurs and instances in which it does not.

“Pharmaceutically acceptable salt” refers to pharmaceutically acceptable salts of fullerenes which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

As used herein, “pharmaceutically acceptable” means acceptable for use in the pharmaceutical and veterinary arts, compatible with other ingredients of the formulation, and not toxic or otherwise unacceptable commensurate with a reasonable benefit/risk ratio.

A “pharmaceutically acceptable carrier” or “diluent” includes any and all solvents, dispersion media, coatings, antibacterial and anti-fungal agents, isotonic and absorption delaying agents, and the like, compatible with pharmaceutical administration of a composition comprising ferritin-iron complexes. Examples of such carriers or diluents include, but are not limited to, water, saline, Ringer's solutions and dextrose solution. The volume of a pharmaceutical composition or formulation comprising fullerenes is based on the intended mode of administration and the safe volume for the individual patient, as determined by a medical professional.

The selection of carrier also depends on the intended mode of administration. Fullerenes of the present invention may be administered by any of a number of convenient means including, but not limited to systemic administration (e.g., intravenous injection, intraparenteral injection, inhalation, transdermal delivery, oral delivery, nasal delivery, rectal delivery, etc.) and/or local administration (e.g., direct injection into a target tissue, delivery into a tissue via cannula, delivery into a target tissue by implantation of a time-release material, or delivery through the skin via a topical composition such as a cream, lotion, or the like), delivery into a tissue by a pump, etc., orally, parenterally, intraosseously, in the cerebrospinal fluid, or the like. Further modes of administration include buccal, sublingual, vaginal, subcutaneous, intramuscular, or intradermal administration.

In some embodiments, a pharmaceutical composition or formulation comprising fullerenes is administered orally to a subject having a wound. As used herein, “pharmaceutical composition” and “pharmaceutical formulation” are interchangeable. In another embodiment, a composition comprising fullerenes is injected directly into an affected area of the body of a subject having a wound. In yet another embodiment, a composition comprising fullerenes is administered via a topical formulation applied to the skin proximal to an affected area of the body of a subject having a wound.

A “therapeutically effective amount” or “pharmaceutically effective amount” means the amount of a fullerene that, when administered to a subject for treating wounds effectively treats the wounds. Thus a “therapeutically effective amount” is an amount indicated for treatment while not exceeding an amount which may cause significant adverse effects. The “therapeutically effective amount” will vary depending on the types of fullerenes to be administered, the severity of the wound, and the age, weight, etc., of the subject to be treated. Methods for evaluating the effectiveness of therapeutic treatments are known to those of skill in the art.

Doses to be administered are variable according to the treatment period, frequency of administration, the host, and the nature and severity of the disorder. The dose can be determined by one of skill in the art without an undue amount of experimentation. The fullerenes are administered in dosage concentrations sufficient to ensure the release of a sufficient dosage unit into the patient to provide the desired treatment of the wound. The actual dosage administered will be determined by physical and physiological factors such as age, body weight, severity of condition, and/or clinical history of the patient. The fullerenes may be administered to achieve in vivo plasma concentrations of the fullerenes of from about 0.01 to 10,000 ng/cc. For example, the methods described in this disclosure may use compositions to provide from about 0.01 to about 100 or from about 0.01 to about 10 mg/kg body weight/day of the fullerenes, such as about 50 mg/kg body weight/day of the fullerenes. It will be understood, however, that dosage levels that deviate from the ranges provided may also be suitable in the treatment of a given disorder. A practical dosage regimen is a schedule of drug administration that is practical for a patient to comply with.

The fullerenes may be in any form suitable for administration. Such administrable forms include tablets, buffered tablets, pills, capsules, enteric-coated capsules, dragees, cachets, powders, granules, aerosols, liposomes, suppositories, creams, lotions, ointments, skin patches, parenterals, lozenges, oral liquids such as suspensions, solutions and emulsions (oil-in-water or water-in-oil), ophthalmic liquids and injectable liquids, or sustained-release forms thereof. The desired dose may be provided in several increments at regular intervals throughout the day, by continuous infusion, or by sustained release formulations, or may be presented as a bolus, electuary or paste.

In various embodiments, a pharmaceutical composition or formulation comprising the fullerenes is prepared by admixture with one or more pharmaceutically acceptable carriers and/or excipients. Other additives and/or active ingredients may be added, if desired, to maximize the preservation of the fullerenes, to optimize a particular method of delivery, or to optimize treatment of wounds in a subject in need thereof. In addition, according to other embodiments, the pharmaceutical composition or formulation comprising fullerenes may include other compositions comprising fullerenes as described herein in combination with other agents suitable for treatment of wounds in the subject in need thereof.

In one embodiment, a kit for treating wounds is disclosed. The kit for treating wounds comprises: administering to a subject in need thereof a therapeutically effective amount of a synthetically modified fullerene of the formula Zₘ—F—Yₙ wherein F is a fullerene of formula Cₚ or X@Cₚ, the fullerene having two opposing poles and an equatorial region; Cₚ represents a fullerene cage having p carbon atoms, and X@Cₚ represents such a fullerene cage having a chemical group X within the cage; Z and Y are positioned near respective opposite poles of Cₚ; m=1-5 and Z is a hydrophilic, lipophilic, or amphiphilic chemical moiety; n=1-5 and Y is a lipophilic chemical moiety; p=60-200 and p is an even number; and X, if present, represents one or more metal atoms within the fullerene (F), optionally in the form of a trinitride of formula Gᵢ₌₁₋₃Hₖ₌₃₋ᵢN in which G and H are metal atoms.

The fullerenes may be formulated into a variety of compositions (i.e., formulations or preparations). These compositions may comprise any component that is suitable for the intended purpose, such as conventional physiologically acceptable delivery vehicles, diluents and excipients including isotonising agents, pH regulators, solvents, solubilizers, dyes, gelling agents and thickeners and buffers and combinations thereof. Pharmaceutical formulations suitable for use with the instant fullerenes can be found, for instance, in Remington's Pharmaceutical Sciences. Physiologically acceptable carriers are carriers that are nontoxic at the dosages and concentrations employed. Pharmaceutical formulations herein comprise pharmaceutical excipients or carriers capable of directing the fullerenes to the area where the subject in need thereof has a wound. Suitable excipients for use with fullerenes include water, saline, dextrose, glycerol and the like.

In various embodiments, the fullerenes are administered to a subject in need thereof in the form of pharmaceutical compositions or formulations. These pharmaceutical compositions or formulations comprise fullerenes and can also include one or more pharmaceutically acceptable carriers or excipients. The excipient is typically one suitable for administration to human subjects or other mammals. In making the compositions of this disclosure, the active ingredient (i.e., fullerenes) is usually mixed with an excipient, and/or diluted by an excipient. When the excipient serves as a diluent, it can be a solid, semi-solid, or liquid material, which acts as a vehicle, carrier or medium for the active ingredient. For additional information regarding suitable methods and formulations for use in the present disclosure are found in REMINGTON'S PHARMACEUTICAL SCIENCES, Mace Publishing Company, Philadelphia, Pa., 17th ed. (1985).

According to one embodiment, the fullerenes may be administered alone, or in combination with any other medicament. Thus, the formulation may comprise fullerenes in combination with another active ingredient, such as a drug, in the same formulation. When administered in combination, the fullerenes may be administered in the same formulation as other compounds as shown, or in a separate formulation. When administered in combination, the fullerenes may be administered prior to, following, or concurrently with the other compounds and/or compositions.

In certain embodiments, the formulations comprise a skin-penetration enhancer. Any skin-penetration enhancer suitable for aiding the delivery of the fullerenes can be used. A list of skin-penetration enhancers can be found in, e.g., “Pharmaceutical Skin Penetration Enhancement” (1993) Walters, K. A., ed.; Hadgraft, J., ed—New York, N.Y. Marcel Dekker and in “Skin Penetration Enhancers cited in the Technical Literature” Osbourne, D. W. Pharmaceutical Technology, November 1997, pp 59-65.

The formulations can comprise from about 0.1% to about 99%, such as from about 0.1% to about 90%, about 5% to about 90%, or about 15% to about 75%, by weight of skin penetration enhancer. In certain embodiments the ratio of fullerenes to skin-penetration enhancer is from about 1:20 to about 1:10000, such as from about 1:60 to 1:300, on the basis of percentages by weight of total composition.

The fullerenes may be solubilized, especially when the fullerenes are hydrophobic. One method of solublizing certain fullerenes is by formulation in liposomes. Other methods suitable for solublizing certain fullerenes include the use of a solvent acceptable for use in the treatment of skin tissues and cells such as, but not limited to, DMSO (dimethylsulfoxide), alcohols, polyethylene glycol (PEG) wherein the size is less than PEG 1000 or any other solvent. Other solublizers include glycol ethers, ethylene glycol, polyethylene glycol derivatives, propylene glycol, propylene glycol derivatives, polysorbates (e.g., TWEEN™), fatty alcohols, aromatic alcohols, propylene glycol, glycerols, oils, surfactants, glucosides, and mixtures thereof. In certain embodiments the solubilizer is selected from diethylene glycol monoethyl ether (TRANSCUTOL®), polyethylene glycol of average molecular weight from 100 to 1000, triethylene glycol, tetraethylene glycol, pentaethylene glycol, hexaethylene glycol, septaethylene glycol, octaethylene glycol, propylene glycol, propylene glycol mono- and diesters of fats and fatty acids (e.g., propylene glycol monocaprylate, propylene glycol monolaurate), benzyl alcohol, glycerol, oleyl alcohol, mineral oil, lanolin/lanolin derivatives, petrolatum or other petroleum products suitable for application to the skin, propylene glycol mono- and diesters of fats and fatty acids, macrogols, macrogolglycerides or polyethylene glycol glycerides and fatty esters (e.g., stearoyl macrogolglycerides, oleoyl macrogolglycerides, lauroyl macrogolglycerides, linoleoyl macrogolglycerides), ethoxylated castor oil (e.g., Cremophor--a polyoxyl hydrogenated castor oil), C6-C30 triglycerides, natural oils, glucosides (e.g., cetearyl glucoside), surfactants, and mixtures thereof. In certain embodiments the formulations herein comprise from about 0.1% to about 99% by weight of solubilizer, such as from about 1% to about 75% by weight of solubilizer.

In certain embodiments the pharmaceutical compositions or formulations described herein have a viscosity at 20° C. of from about 5 cps to about 50000 cps, such as from about 500 cps to about 40000 cps, or about 5000 cps to about 30000 cps. Should the viscosity need to be adjusted it can be done by means of a viscosity modifying agent. Examples of viscosity modifiers include polyethylene glycols, acrylic acid-based polymers (carbopol polymers or carbomers), polymers of acrylic acid crosslinked with allyl sucrose or allylpentaerythritol (carbopol homopolymers), polymers of acrylic acid modified by long chain (C₁₀-C₃₀) alkyl acrylates and crosslinked with allylpentaerythritol (carbopol copolymers), poloxamers also known as pluronics (block polymers; e.g., Poloxamer 124, 188, 237, 338, 407), waxes (paraffin, glyceryl monostearate, diethylene glycol monostearate, propylene glycol monostearate, ethylene glycol monostearate, glycol stearate), hard fats (e.g., Saturated C₈-C₁₈ fatty acid glycerides), xantham gum, polyvinyl alcohol, solid alcohols, and mixtures thereof.

In certain embodiments the formulations contain one or more PEGs. Examples include at least one PEG of average molecular weight about 2000 or less, about 1500 or less, about 1000 or less, about 800 or less, about 600 or less, about 500 or less, or about 400 or less. Examples also include at least one PEG of average molecular weight about 3000 or more, about 3350 or more, or about 3500 or more. In one embodiment the formulation comprises a mixture of PEG's, such as at least one PEG having an average molecular weight of about 800 or less and at least one PEG having an average molecular weight of 3000 or more.

The formulation may comprise a variety of other components. Any suitable ingredient may be used herein but typically these optional component will render the formulations more cosmetically acceptable or provide additional usage benefits. Some examples of optional ingredients include, but are not limited to, emulsifiers, humectants, emollients, surfactants, oils, waxes, fatty alcohols, dispersants, skin-benefit agents, pH adjusters, dyes/colorants, analgesics, perfumes, preservatives, and mixtures thereof. In addition, the methods described herein include use of combination compositions comprising the fullerenes as described herein in combination with other agents suitable for treating wounds in a subject in need thereof.

Examples of suitable preservatives include but are not limited to parabens, benzyl alcohol, quaternium 15, imidazolidyl urea, disodium EDTA, methylisothiazoline, alcohols, and mixtures thereof. Examples of suitable emulsifiers include but are not limited to waxes, sorbitan esters, polysorbates, ethoxylated castor oil, ethoxylated fatty alcohols, macrogolglycerides or polyethylene glycol glycerides and fatty esters (e.g., stearoyl macrogolglycerides, oleoyl macrogolglycerides, lauroyl macrogolglycerides), esters of saturated fatty acids (e.g., diethylene glycol parmitostearate), macrogols of cetostearyl ether (e.g., macrogol-6-cetostearyl ether), polymers of high molecular weight, crosslinked acrylic acid-based polymers (carbopols or carbomers), and mixtures thereof. Examples of suitable emollients include but are not limited to propylene glycol dipelargonate, 2-octyldodecyl myristate, non-polar esters, triglycerides and esters (animal and vegetable oils), lanolin, lanolin derivatives, cholesterol, glucosides (e.g., cetearyl glucoside), pegylated lanolin, ethoxylated glycerides, and mixtures thereof Examples of suitable surfactants include but are not limited to sorbitan esters, polysorbates, sarcosinates, taurate, ethoxylated castor oil, ethoxylated fatty alcohols, ethoxylated glycerides, caprylocaproyl macrogol-8 glycerides, polyglyceryl-6 dioleate, and mixtures thereof Examples of suitable oils include but are not limited to propylene glycol monocaprylate, medium chain triglycerides (MCT), 2-octyl-dodecyl myristate, cetearyl ethylhexanoate, and mixtures thereof. Examples of suitable fatty alcohols include but are not limited to cetostearyl alcohol, cetyl alcohol, stearyl alcohol, and mixtures thereof. Also useful in the formulations herein are lipids and triglycerides (e.g., concentrates of Seed Oil Lipids, Concentrates of Marine Oil Lipids, high purity triglycerides and esters), alkyl ether sulfates, alkyl polyglycosides, alkylsulfates, amphoterics cream bases, and mixtures thereof.

Preparation of dry formulations that are reconstituted immediately before use also is contemplated. The preparation of dry or lyophilized formulations can be effected in a known manner, conveniently from the solutions of the invention.

The dry formulations of this invention are also storable. By conventional techniques, a solution can be evaporated to dryness under mild conditions, especially after the addition of solvents for azeotropic removal of water, typically a mixture of toluene and ethanol. The residue is thereafter conveniently dried, e.g., for some hours in a drying oven.

The method herein is targeted to a widespread or localized area on the body of a subject having a wound. The fullerene-containing preparations described above may be administered systemically or locally and may be used alone or as components of mixtures. In one embodiment the administration is local. The route of administration for the fullerenes may be topical, intradermal, intravenous, oral, or by use of an implant. In one embodiment the route of administration is topical. For example, fullerenes may be administered by means including, but not limited to, topical lotions, topical creams, topical pastes, topical suspensions, intravenous injection or infusion, oral intake, or local administration in the form of intradermal injection or an implant. Additional routes of administration are subcutaneous, intramuscular, or intraperitoneal injections of the fullerenes in conventional or convenient forms.

For topical formulations (such as ointments) to be applied to the surface of the skin, the concentration of the fullerenes in the excipient preferably ranges from about 0.001 to about 10% w/w, such as from about 0.005 to about 5% w/w, or from about 0.01 to about 1% w/w. The foregoing ranges are merely suggestive in that the number of variables with regard to an individual treatment regime is large and considerable deviation from these values may be expected.

When administered topically, the area to be treated may be massaged after application of the fullerenes.

Suitable isotonising agents are for example nonionic isotonising agents such as urea, glycerol, sorbitol, mannitol, aminoethanol or propylene glycol as well as ionic isotonising agents such as sodium chloride. Solutions containing fullerenes will contain the isotonising agent, if present, in an amount sufficient to bring about the formation of an approximately isotonic solution. The expression “an approximately isotonic solution” will be taken to mean in this context a solution that has an osmolarity of about 300 milliosmol (mOsm), conveniently 300+10% mOsm.

It should be borne in mind that all components of the solution contribute to the osmolarity. The nonionic isotonising agent, if present, is added in customary amounts, i.e., preferably in amounts of about 1 to about 3.5 percent by weight, such as in amounts of about 1.5 to 3 percent by weight.

In one embodiment, the fullerenes are delivered topically. For topical administration, the fullerenes may be in standard topical formulations and compositions including lotions, creams, suspensions, serums, or pastes. Solubilized fullerenes can also be added to other dermatological products, such as hair gels, shampoos, conditioners, styling products, soaps, or the like. Injection may also be used when desired. Oral administration of suitable formulations may also be appropriate in those instances where the fullerenes may be readily administered to the widespread or localized area(s) on the body of the subject having a wound via this route.

Generally, the pharmaceutical compositions or formulations described herein can be administered as a pharmaceutical, cosmetic, or nutritional formulation. These compositions or formulations can be administered topically, orally, intravenously, or as a suppository.

The present disclosure relates to use of any one or more of the fullerenes described herein for the treatment of wounds. The present disclosure also relates to the use of any one or more of the fullerenes described herein for manufacture of a medicament, particularly the manufacture of a medicament for treating wounds.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates, which may need to be independently confirmed. All publications, patents, patent applications and other references cited herein are hereby incorporated by reference.

While the disclosure has been described in detail with reference to certain embodiments thereof, it will be apparent to one skilled in the art that various changes can be made, and equivalents employed, without departing from the scope of the disclosure. In addition, the following examples are illustrative of the methods described herein and should not be considered as limiting the foregoing disclosure in any way.

### EXAMPLES

### Example 1

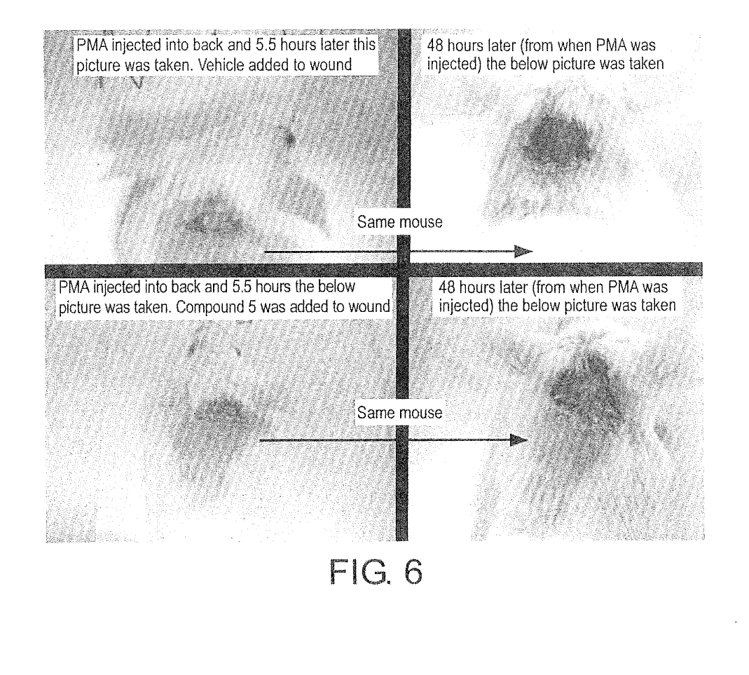

Compound 5 (ALM) (illustrated in FIG. 1) was tested for the ability to prevent the initial inflammatory response in the wound healing process in a mouse model of irritant dermatitis wound induction. Acetone (20 μl Wear) or compound 5 (ALM) (2 μg/20 μl) were injected subcutaneously in the left ear 2-5 minutes prior to injection with Phorbol myristilic acid (PMA) (20 μl of 0.01% w/v per ear) or acetone (20 μl per right ear) of Balb/c mice. See FIG. 6. The right ear, which serves as a control, was injected with equal volumes of vehicle alone. After 5.5 hours the PMA treated ears exhibit swelling and erythema (FIG. 6—left) which is dramatically inhibited when fullerenes are injected prior to PMA challenge (FIG. 6 -right). The extent of swelling was quantitated by weighing a 3 mm biopsy from the left (PMA) and right (vehicle only) ears. These results show that compound 5 (ALM) effective in inhibiting the induction of the PMA challenged ear wound.

### Example 2

The next investigation was to determine if compound 5 (ALM) could accelerate the healing of a wound. The intrascapular part of the back of Balb/c mice were shaved 1 day prior to each experiment. PMA (300 μg/50 μl PBS) was injected intradermally. After 5.5 hours the wound was photo-documented before injection with compound 5 (ALM) (100-1000 ng/50 μl) or PBS. After at least 24 hours mice were sacrificed and the degree of wound healing compared between control and treated mice. As seen in FIG. 7 the wound that was injected with compound 5 (ALM) healed much faster compared to the wound injected with vehicle only.

### Example 3

Wound healing can be modeled in tissue culture. In this experiment, 3T3 mouse fibroblasts were grown to confluence in tissue culture dishes by standard practices. Once a monolayer covered the surface of the tissue culture dish a wound was inflicted on the monolayer by scraping a line across the culture dish under aseptic conditions. Those 3T3 cells in the vicinity of the scratch infiltrated and grew into the wound to restore confluence.

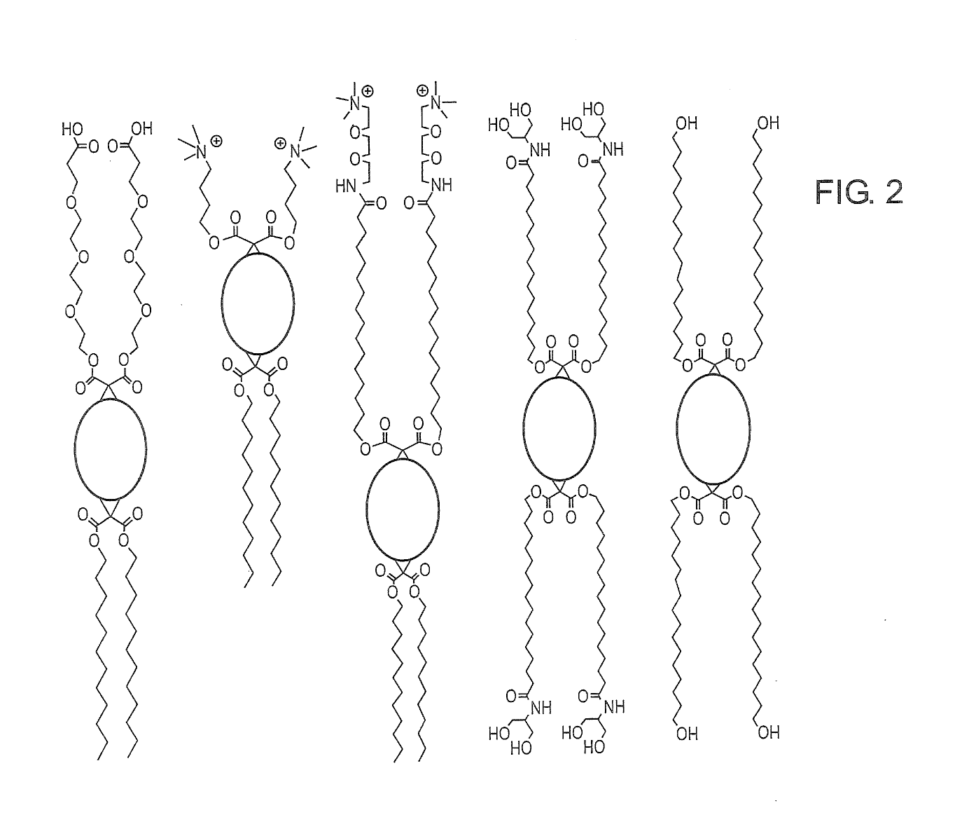

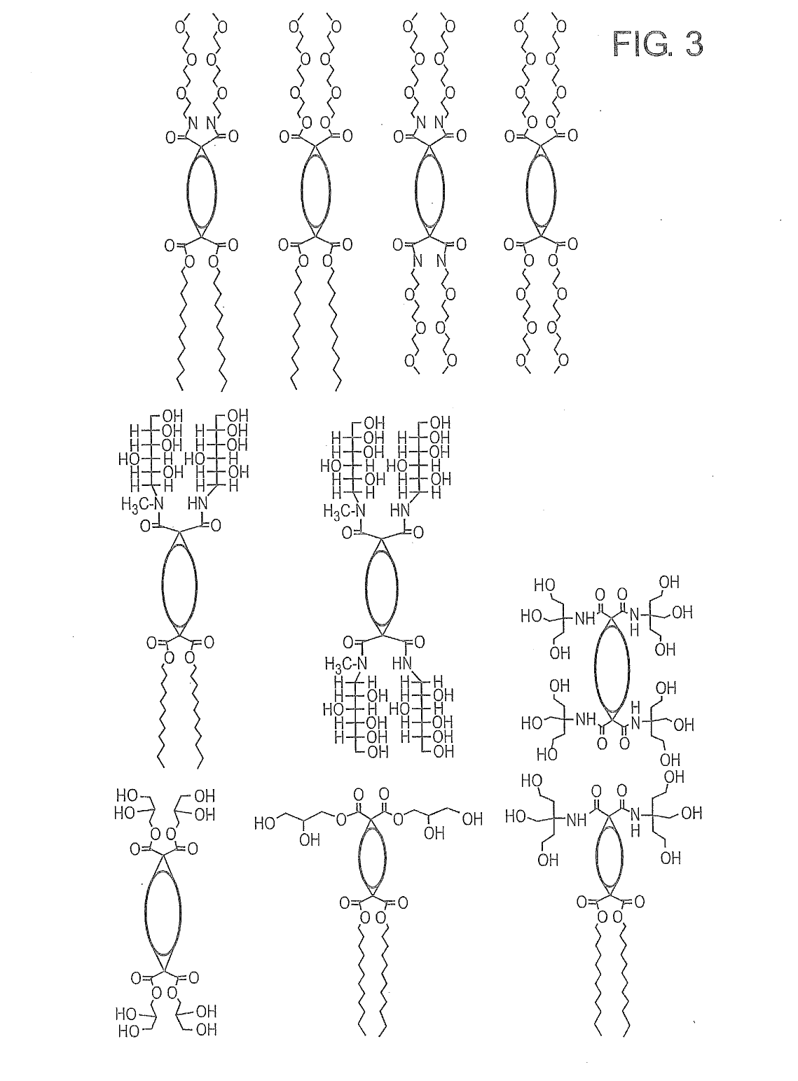

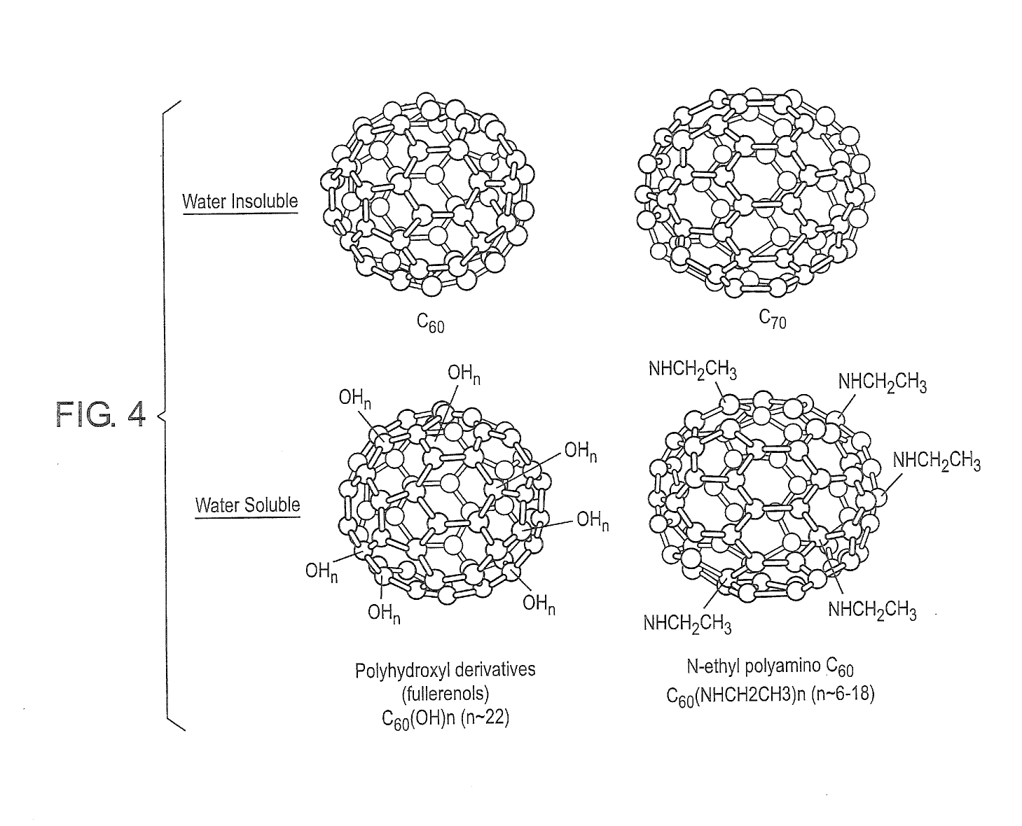

The rate at which such wounds repair the scrape is used to predict the wound healing properties of therapeutic candidates. All of the compounds illustrated in FIGS. 1-4 accelerated the regrowth of fibroblasts monolayers (data not shown).

While various embodiments have been particularly shown and described herein, it will be understood by those skilled in the art that various changes in form and details may be made therein without departing from the spirit and scope of these embodiments as further defined by the appended claims.

1. A method for treating a wound, comprising:
administering to a subject in need thereof a therapeutically effective amount of a synthetically modified fullerene of the formula
Zₘ—F—Yₙ
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage;
Z and Y are positioned near respective opposite poles of Cₚ;
m=1-5 and Z is a hydrophilic, lipophilic, or amphiphilic chemical moiety;
n=1-5 and Y is a lipophilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms.

2. The method of claim 1, wherein p is an even number between 60 and 120.

3. The method of claim 2, wherein p is an even number between 60 and 96.

4. The method of claim 3, wherein p is 60 or 70.

5. The method of claim 4, wherein p is 70.

6. The method of claim 1, wherein said synthetically modified fullerene is a prolate ellipsoid shaped fullerene having a major axis such that said poles are located at opposing ends of the major axis of the prolate ellipsoid fullerene.

7. The method of claim 1, wherein said synthetically modified fullerene is spheroid with opposing poles defined by an axis through opposing carbon rings.

8. The method of claim 1, wherein said subject is a human.

9. The method of claim 1, wherein said synthetically modified fullerene is administered as a pharmaceutical composition comprising at least one carrier and/or at least one excipient.

10. The method of claim 1, wherein said synthetically modified fullerene is administered topically, orally, intravenously, or as a suppository.

11. The method of claim 9, wherein said synthetically modified fullerene is administered in the form of a cream.

12. The method of claim 1, wherein said synthetically modified fullerene is administered to said subject in combination with at least one other active ingredient.

13. The method of claim 1, wherein said wound is widespread or localized.

14. A method for treating a wound, comprising:
administering to a subject in need thereof a therapeutically effective amount of a synthetically modified fullerene of the formula
Z(Cₚ)Y
wherein p=60-200 carbons, preferably p=60 or 70; Y is a lipophilic moiety covalently connected to Cₚ, optionally through a linking group, at or near a pole thereof, and wherein Z is a lipophilic moiety, amphiphilic moiety, or a hydrophilic moiety covalently connected to Cₚ, optionally through a linking group, at or near a pole opposite to said Y.

15. The method of claim 14, wherein Cₚ is C₇₀.

16. The method of claim 14 or claim 15, wherein Z is a hydrophilic moiety.

17. The method of claim 14, wherein the synthetically modified fullerene is compound 5 (ALM).

18. The method of claim 14, wherein said subject is a human.

19. The method of claim 14, wherein said synthetically modified fullerene is administered as a pharmaceutical composition comprising at least one carrier and/or at least one excipient.

20. The method of claim 14, wherein said synthetically modified fullerene is administered topically, orally, intravenously, or as a suppository.

21. The method of claim 15, wherein said synthetically modified fullerene is administered in the form of a cream.

22. The method of claim 14, wherein said synthetically modified fullerene is administered to said subject in combination with at least one other active ingredient.

23. The method of claim 14, wherein said wound is widespread or localized.

24. A method for treating a wound, comprising:
administering to a subject in need thereof a therapeutically effective amount of a synthetically modified fullerene of the formula
Z′ₘ—F—Y′ₙ;
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage;
Z′ and Y′ are positioned near respective opposite poles of Cₚ;
m=1-5 and Z′ is a hydrophilic, lipophilic, or amphiphilic chemical moiety;
n=1-5 and Y′ is a hydrophilic or amphiphilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms.

25. The method of claim 24, wherein p is an even number between 60 and 120.

26. The method of claim 25, wherein p is an even number between 60 and 96.

27. The method of claim 26, wherein p is 60 or 70.

28. The method of claim 27, wherein p is 70.

29. The method of claim 24, wherein said synthetically modified fullerene is a prolate ellipsoid shaped fullerene having a major axis such that said poles are located at opposing ends of the major axis of the prolate ellipsoid fullerene.

30. The method of claim 24, wherein said synthetically modified fullerene is spheroid with opposing poles defined by an axis through opposing carbon rings.

31. The method of claim 24, wherein
Z′ comprises the formula A′,B wherein A′ is a hydrophilic, lipophilic or amphiphilic chemical moiety, r=1-4, and B is a chemical linker connecting said A′ to the fullerene;
Y′ comprises the formula DE′ᵥ wherein E′ is a hydrophilic or amphiphilic chemical moiety and, v=1-4, and D is a chemical linker connecting the chemical moiety Y′ to the fullerene.

32. The method of claim 24, wherein said subject is a human.

33. The method of claim 24, wherein said synthetically modified fullerene is administered as a pharmaceutical composition comprising at least one carrier and/or at least one excipient.

34. The method of claim 24, wherein said synthetically modified fullerene is administered topically, orally, intravenously, or as a suppository.

35. The method of claim 34, wherein said synthetically modified fullerene is administered in the form of a cream.

36. The method of claim 24, wherein said synthetically modified fullerene is administered to said subject in combination with at least one other active ingredient.

37. The method of claim 24, wherein said wound is widespread or localized.

38. A method for treating a wound, comprising:
administering to a subject in need thereof a therapeutically effective amount of a synthetically modified fullerene of the formula
Z′(Cₚ)Y′
wherein: p=60-200 carbons, preferably p=60 or 70; Y′ is a hydrophilic or amphiphilic moiety covalently connected to Cₚ, optionally through a linking group, at or near a pole thereof, and wherein Z′ is a hydrophilic or amphiphilic moiety covalently connected to Cₚ, optionally through a linking group, at or near a pole opposite to said Y′.

39. The method of claim 38, wherein
(a) Z′ and Y′ are both amphiphilic;
(b) Z′ and Y′ are both hydrophilic;
(c) one of Z′ and Y′ is amphiphilic while the other is hydrophilic;
(d) Z′ is lipophilic and Y′ is hydrophilic; or
(e) Z′ is lipophilic and Y′ is amphiphilic.

40. The method of claim 39, wherein Z′ and Y′ are both hydrophilic.

41. The method of claim 40, wherein Cₚ=C₇₀.

42. The method of claim 41, wherein the synthetically modified fullerene is compound 7 (TGA).

43. The method of claim 38, wherein said subject is a human.

44. The method of claim 38, wherein said synthetically modified fullerene is administered as a pharmaceutical composition comprising at least one carrier and/or at least one excipient.

45. The method of claim 38, wherein said synthetically modified fullerene is administered topically, orally, intravenously, or as a suppository.

46. The method of claim 38, wherein said synthetically modified fullerene is administered in the form of a cream.

47. The method of claim 38, wherein said synthetically modified fullerene is administered to said subject in combination with at least one other active ingredient.

48. The method of claim 38, wherein said wound is widespread or localized.

49. A method for treating a wound, comprising administering to a subject in need thereof a therapeutically effective amount of a synthetically modified fullerene, wherein the synthetically modified fullerene is any one or more of the compounds shown in the present figures.

50. The method of claim 49, wherein the synthetically modified fullerene is selected from the group consisting of compound 5 (ALM), compound 7 (TGA), and combinations thereof.

51. A synthetically modified fullerene of the formula
Zₘ—F—F—Yₙ
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage.
Z and Y are positioned near respective opposite poles of Cₚ;
m=1-5 and Z is a hydrophilic, lipophilic, or amphiphilic chemical moiety;
n=1-5 and Y is a lipophilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms, for use in treating a wound.

52. A synthetically modified fullerene of the formula
Zₘ—F—Yₙ
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage.
Z and Y are positioned near respective opposite poles of Cₚ;
m=1-5 and Z is a hydrophilic, lipophilic, or amphiphilic chemical moiety;
n=1-5 and Y is a lipophilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms, for preparation of a medicament for treating a wound.

53. A synthetically modified fullerene of the formula
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage;
Z′ and Y′ are positioned near respective opposite poles of Cₚ;
m=1-5 and Z′ is a hydrophilic, lipophilic, or amphiphilic chemical moiety;
n=1-5 and Y′ is a hydrophilic or amphiphilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms, for use in treating a wound.

54. A synthetically modified fullerene of the formula
Z′ₘ—F—Y′ₙ;
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage;
Z′ and Y′ are positioned near respective opposite poles of Cₚ;
m=1-5 and Z′ is a hydrophilic, lipophilic, or amphiphilic chemical moiety;
n=1-5 and Y′ is a hydrophilic or amphiphilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms, for preparation of a medicament for treating a wound.
